(19) United States
(12) Patent Application Publication
(10) Pub. No.: US 2003/0044462 A1
(43) Pub. Date: Mar. 6, 2003

(54) Sustained release tablets containing bupropion hydrochloride

(75) Inventors: Veerappa S. Subramanian, Edison, NJ (US); Muthusamy Shanmugam, Edison, NJ (US)

Correspondence Address: Kent H. Cheng, Esq., Cohen, Pontani, Lieberman & Pavane, Suite1210, 551 Fifth Avenue, New York, NY, 10176, US

(73) Assignee: Kali Laboratories, Inc.

(21) Appl. No.: 09/933,559

(22) Filed: Aug. 20, 2001

(51) Int. Cl. A61K 31/135; A61K 9/22

(52) U.S. Cl. 424/468; 514/649

(57) ABSTRACT

This application describes a method of preparing stabilized sustained release tablets containing bupropion hydrochloride and carboxyvinyl polymers, in which the composition contains, at least about 90% w/w of undegraded Bupropion hydrochloride after storage for two weeks at 55° C. and for three months at 40° C. and 75% relative humidity.

### BACKGROUND OF THE INVENTION

1. Field of the Invention

Bupropion hydrochloride is a known antidepressant, which is marketed as a sustained release tablet form under the brand name of Wellbutrin® by Glaxo Wellcome, Inc. It is chemically known as 1-(3-chlorophenyl)-2-[(1,1-dimethylethyl)-amino]-1-propanone hydrochloride (see U.S. Pat. Nos. 3,819,706 and 3,885,046, and Merck Index, Eleventh Edition, entry no. 1488). Bupropion hydrochloride is a stability prone product when formulated with conventional pharmaceutical excipients into solid dosage form. Though exact mechanism of degradation has not been fully elucidated, literature and patent information seem to indicate that hydrolysis and oxidation are the possible mechanisms of degradation. The main degradation product is 3-chlorobenzoic acid.

U.S. Pat. Nos. 5,541,231; 5,358,970 and 5,731,000 disclose that ascorbic acid, isoascorbic acid, L-Cysteine hydrochloride, glycine hydrochloride, malic acid, citric acid, fumaric acid, sodium metabisulfite, and L-Cysteine dihydrochloride inhibit the degradation of bupropion hydrochloride in pharmaceutical preparations. U.S. Pat. No. 5,968,553 discloses a novel tablet formulation comprising bupropion hydrochloride and inorganic acid stabilizers selected from the group consisting of hydrochloric acid, phosphoric acid, nitric acid and sulfuric acid. U.S. Pat. No. 5,427,798 discloses sustained release tablets containing bupropion hydrochloride with use of cellulose polymer (hydroxy propylmethyl cellulose), which provides an improved product as well as ease of manufacture over prior U.S. Pat. No. 4,687,660.

Currently, marketed immediate release tablets, for example, the 75 mg and 100 mg, when administered to humans for treatment of depression, are given one to three times per day in order to provide a total daily dosage of 150 mg to 450 mg for the duration of treatment as determined by the physician. The present invention would reduce the frequency of dosing, and thus enhance compliance of dosage regimen, since it would provide for a possible one or two times a day administration, rather than the three times per day. Modified release and extended release dosage forms are encompassed by the present invention.

2. Detailed Description of the Invention

The invention is not limited by the embodiments described above which are presented as examples only but can be modified in various ways within the scope of protection defined by the appended patent claims.

The object of the present invention is to prevent or inhibit the degradation of bupropion hydrochloride by use of carboxyvinyl polymer as stabilizers in an effective amount in which the composition contains, at least, about 90% w/w of undegraded bupropion hydrochloride after storage for two weeks at 55° C., and for three months at 40° C. and 75% relative humidity. A carboxyvinyl polymer is an interpolymer of a monomeric mixture comprising a monomeric olefinically unsaturated carboxylic acid. Various carboxyvinyl polymers are commercially available from B.F. Goodrich Company, New York, N.Y., under the trade name of Carbopol®. The primary functions of the carboxyvinyl polymer in the sustained release tablets of the present invention are to stabilize bupropion hydrochloride, and control the duration of the sustained release of bupropion hydrochloride. As the concentration of carboxyvinyl polymer is increased, the duration of sustained release of bupropion hydrochloride increases; therefore, the percentage of carboxyvinyl polymer is adjusted to achieve the desired sustained release rate of bupropion hydrochloride.

The present invention also provides for a pharmaceutical composition designed for sustained release (SR) tablets, containing bupropion hydrochloride and carboxyvinyl polymer (Carbopol®) and other pharmaceutically acceptable excipients, preferably lactose and microcrystalline cellulose for controlling the rate of release of the active ingredient for twice a day and once a day dosage regimen. The amount of bupropion hydrochloride present in each tablet is typically 150 mg and 200 mg, respectively, although other strengths are operable. The tablets may be film coated (with color for product identification) for taste masking and/or aesthetic appearance. Such film coats are non-functional, i.e., they do not adversely affect the release characteristics of bupropion hydrochloride from the tablets.

The effective amount of Carbopol® that could be used in the present invention to achieve stability and sustained release of bupropion hydrochloride may vary between 0.5% and 30%, but preferably between 6% and 28% w/w and most preferably between 10% and 28% of the tablet weight. In general, any amount that will effectively retard or prevent degradation of bupropion hydrochloride, and demonstrate a sustained release profile of the active drug, can be used.

The release rate of bupropion hydrochloride from the sustained release tablets disclosed in the present invention, when determined using the United States Pharmacopoeia apparatus II (rotating paddle) at 50 rpm in 900 mL distilled water at 37° C.±0.5° C. is as follows: about 30-45% is released in 1 hour, between 60 and 80% is released in 4 hours, and not less than 85% is released in 7 hours for the twice a day product, and about 10-25% in 1 hour, 30-60% in 8 hours and not less than 65% in 12 hours.

The composition of the present invention may be prepared by the wet granulation method well known in the art of pharmacy. In general, the compositions are blended in a high shear granulator, and granulated with purified water as granulating agent. After drying and milling, the blend is lubricated with a suitable lubricant, such as magnesium stearate, and compressed into core tablets. The core tablets are then film coated for aesthetic appearance and/or taste masking.

The following examples are representative of the present invention.

### EXAMPLE 1

### (Ref: -03-095)

Stable bupropion hydrochloride and sustained release formulation can be made containing the following ingredients:

| | Quantity/Unit |
|---|---|
| Ingredients | (mg) |
| Bupropion Hydrochloride | 150.0 |
| Carbopol ® 971P, NF | 40.20 |
| Lactose Monohydrate NF (Spray Dried) | 207.60 |
| Purified Water | q.s. |
| Magnesium Stearate, NF | 4.20 |
| Total Weight | 402.00 |

Bupropion hydrochloride, Carbopol® and lactose monohydrate were mixed in a high shear granulator and granulated with purified water as the granulating agent. After drying in an oven and milling the dried granules, the blend was lubricated with magnesium stearate, and compressed into core tablets.

The compressed tablet cores (30,000) were aqueous film coated using the following coating formulation.

| | |
|---|---|
| Opadry Red, YS-1-1846 | 300.0 g |
| Purified Water, USP | 2,200.0 g |
| | 2,500.0 g |

The Purified Water was added to a stainless steel container, and Opadry Red slowly added to the same container with stirring. The stirring was continued until a uniform and smooth suspension was produced. The tablet cores were then coated using a perforated coating pan.

### EXAMPLE 2

### (Ref: -03-175)

The procedure for Example 1 was repeated, except that the amount of Carbopol® was increased, resulting in the following formulation.

| | Quantity/Unit |
|---|---|
| Ingredients | (mg) |
| Bupropion Hydrochloride | 150.0 |
| Carbopol ® 971P, NF | 48.30 |
| Lactose Monohydrate, NF (Spray Dried) | 199.50 |
| Purified Water, USP | q.s. |
| Magnesium Stearate, NF | 4.20 |
| Total Weight | 402.0 |

### EXAMPLE 3

### (Ref: -03-173)

The procedure for Example 1 was repeated, except that the amount of Carbopol® was increased, resulting in the following formulation.

| | Quantity/Unit |
|---|---|
| Ingredients | (mg) |
| Bupropion Hydrochloride | 150.0 |
| Carbopol ® 971P, NF | 60.30 |
| Lactose Monohydrate, NF (Spray Dried) | 187.50 |
| Purified Water, USP | q.s. |
| Magnesium Stearate, NF | 4.20 |
| Total Weight | 402.08 |

### EXAMPLE 4

### (Ref: -037-016)

One preferred composition to be used in accordance with the invention contains the following ingredients in the following amounts.

| | Quantity/Unit |
|---|---|
| Ingredients | (mg) |
| Bupropion Hydrochloride | 150.0 |
| Microcrystalline Cellulose, NF | 187.50 |
| Carbopol ® 971P, NF | 40.00 |
| Purified Water, USP | q.s. |
| Carbopol ® 971P, NF | 20.30 |
| Magnesium Stearate, NF | 4.20 |
| Total Weight | 402.00 |

Bupropion hydrochloride, microcrystalline cellulose and Carbopol® were mixed in a high shear mixer/granulator and granulated with purified water as the granulating agent. After drying in an oven, the dried granules were milled. The milled granules were placed in a twin-shell blender and mixed with the extragranular Carbopol®. The blend was lubricated with magnesium stearate, compressed into tablet cores and film coated following the procedure as described for Example 1.

### EXAMPLE 5

### (Ref: -037-019)

A more highly preferred composition to be used in accordance with the invention contains the following ingredients in the following amounts.

| | Quantity/Unit |
|---|---|
| Ingredients | (mg) |
| Bupropion Hydrochloride | 150.0 |
| Carbopol ® 971P NF | 32.20 |
| Lactose Monohydrate, NF (Spray Dried) | 185.90 |
| Purified Water, USP | q.s. |
| Carbopol ® 971P, NF | 28.10 |
| Colloidal Silicon Dioxide, NF | 1.60 |
| Magnesium Stearate, NF | 4.20 |
| Total Weight | 402.00 |

Bupropion hydrochloride, Carbopol® and lactose monohydrate were mixed in a high shear mixer/granulator, and granulated with purified water. After drying in an oven, the dried granules were milled. The milled granules were placed in a twin-shell blender and mixed with the extragranular Carbopol® and colloidal silicon dioxide. The blend was lubricated with magnesium stearate, compressed into tablet cores and film coated following the procedure as described for Example 1.

### EXAMPLE 6

### (Ref: -037-024)

The procedure for Example 4 was repeated, except that the dosage strength and the amount of Carbopol®) were increased to provide a once a day release product, resulting in the following formulation.

| | Quantity/Unit |
|---|---|
| Ingredients | (mg) |
| Bupropion Hydrochloride | 200.00 |
| Microcrystalline Cellulose, NF | 250.00 |
| Carbopol ® 971P, NF | 53.33 |
| Purified Water, USP | q.s. |
| Carbopol ® 971P, NF | 26.67 |
| Magnesium Stearate, NF | 5.60 |
| Total Weight | 535.60 |

### EXAMPLE 7

### (Ref: -037-025)

The procedure for Example 6 was repeated, except that the amount of Carbopol® was increased, resulting in the following formulation.

| | Quantity/Unit |
|---|---|
| Ingredients | (mg) |
| Bupropion Hydrochloride | 200.00 |
| Microcrystalline Cellulose, NF | 179.33 |
| Carbopol ® 971P, NF | 53.33 |
| Purified Water, USP | q.s. |
| Carbopol ® 971P, NF | 26.67 |
| Carbopol ® 71S, NF | 70.72 |
| Magnesium Stearate, NF | 5.60 |
| Total Weight | 536.00 |

### EXAMPLE 8

The stability of the tablets prepared in accordance with the invention was tested at 55° C. for 2 weeks and at 40° C. and 75% relative humidity, and the results are shown as follows.

% Assay

| | | |
|---|---|---|
| | | 40° C. /75% |
| Initial | 2 Weeks | Relative Humidity |

| t = 0 | 55° C. | 1 Month | 3 Months |
|---|---|---|---|
| 97.5 | 100.9 | 97.6 | 99.5 |

We I claim:

1. A pharmaceutical composition in solid form comprising bupropion hydrochloride and carboxyvinyl polymer in an effective stabilizing amount.

2. The composition of claim 1, in which the composition contains at least about 90% w/w of undegraded bupropion hydrochloride after storage for 2 weeks at 55° C.

3. The composition of claim 1, in which the composition contains at least about 90% w/w of undegraded bupropion hydrochloride after storage for 3 months at 40° C. and 75% relative humidity.

4. A pharmaceutical composition according to claim 1, which comprises from about 0.5% to 30% by weight of carboxyvinyl polymer as stabilizer to inhibit the degradation of bupropion hydrochloride.

5. A pharmaceutical composition according to claim 1, which comprises from about 5% to 30% by weight of carboxyvinyl polymer to provide drug release over a period of from about 8 hours to about 24 hours.

6. A pharmaceutical composition according to claim 5, which comprises from about 5% to about 30% by weight of the carboxyvinyl polymer.

7. A pharmaceutical composition according to claim 5, which comprises from about 8% to 28% by weight of carboxyvinyl polymer.

8. A pharmaceutical composition according to claim 5, which comprises from about 10% to about 28% by weight of carboxyvinyl polymer.

9. A method of stabilizing bupropion hydrochloride in a pharmaceutical composition according to claim 1, wherein said method comprises mixing bupropion hydrochloride with suitable pharmaceutical excipients and carboxyvinyl polymer and granulating with purified water.

10. A pharmaceutical composition according to claim 1 further comprising a pharmaceutical excipient selected from the group consisting of lactose, magnesium stearate and microcrystalline cellulose.

11. A pharmaceutical composition according to claim 10, wherein the pharmaceutical excipient is microcrystalline cellulose.

12. A sustained release tablet comprising bupropion hydrochloride, carboxyvinyl polymer and lactose.

13. A sustained release tablet comprising bupropion hydrochloride, carboxyvinyl polymer and microcrystalline cellulose.

14. A sustained release tablet according to claim 12, wherein the mean release of bupropion hydrochloride is one of about between 30% and 45% in 1 hour, about between 60% and 80% in 4 hours, and not less than 85% in 7 hours when tested in distilled water using the United States Pharmacopoeia paddle dissolution method at a rotational speed of 50 rpm.

15. A sustained release tablet according to claim 13, wherein the mean release of bupropion hydrochloride is one of about between 30% and 45% in 1 hour, about between 60% and 80% in 4 hours, and not less than 85% in 7 hours when tested in distilled water using the United States Pharmacopoeia paddle dissolution method at a rotational speed of 50 rpm.

16. A sustained release tablet according to claim 12, wherein the mean release of bupropion hydrochloride is one of about between 10% and 25% in 1 hour, about between 30% and 60% in 8 hours, and not less than 65% in 12 hours when tested in distilled water using the United States Pharmacopoeia paddle dissolution method at a rotational speed of 50 rpm.

17. A sustained release tablet according to claim 13, wherein the mean release of bupropion hydrochloride is one of about between 10% and 25% in 1 hour, about between 30% and 60% in 8 hours, and not less than 65% in 12 hours when tested in distilled water using the United States Pharmacopoeia paddle dissolution method at a rotational speed of 50 rpm.
